(19) United States
(12) Patent Application Publication
(10) Pub. No.: US 2023/0090318 A1
(43) Pub. Date: Mar. 23, 2023

(54) DECOMPRESSIVE GASTROSTOMY TUBE

(71) Applicant: Children's Hospital Medical Center of Akron, Akron, OH (US)

(72) Inventors: Justin Huntington, Hudson, OH (US); Stefan Agamanolis, Akron, OH (US)

(73) Assignee: Children's Hospital Medical Center of Akron, Akron, OH (US)

(21) Appl. No.: 17/947,349

(22) Filed: Sep. 19, 2022

Related U.S. Application Data
(60) Provisional application No. 63/245,396, filed on Sep. 17, 2021.

(51) Int. Cl. A61J 15/00; A61M 1/00

(52) U.S. Cl. CPC A61J 15/0003; A61J 15/0049; A61J 15/0073; A61M 1/71

(57) ABSTRACT

One or more techniques and/or systems are disclosed for a gastrostomy tube having a proximal portion and a distal portion with at least one lumen extending therethrough. The proximal portion includes first, second, and third ports that are each operably coupled to the proximal portion with the first port configured to provide for suction of contents from a patient's stomach, and the third port configured to provide fluid to an inflatable balloon. The distal portion includes a plurality of first openings configured to transport contents to and from the patient's stomach, at least one second opening configured to cooperate with the second port to provide sump-type function, and a curved portion having a gradual bend of approximately 45°-135°. The curved portion directs a distal end of the tube radially outward and away from a longitudinal axis of the tube.

### CROSS-REFERENCE TO RELATED APPLICATIONS

This non-provisional patent application claims priority to provisional application having application No. 63/245,396, filed on Sep. 17, 2021, the entirety of which is incorporated by reference.

### BACKGROUND

Gastrostomy tubes (G-tubes) are devices that allow patients to have enteral nutrition provided directly into their stomach. In particular, G-tubes are feeding tubes that are used to deliver nutrients, fluids, and/or medication directly into a patient's gastro-intestinal tract (e.g., stomach). G-tubes are typically used in patients who do not tolerate oral feeding well. The gastrostomy tube, which is often placed by either a surgeon, gastroenterologist, or an interventional radiologist, typically enters the abdomen, with the distal end of the tube extending directly into the stomach and the proximal end disposed outside the patient's body and accessible by the medical professional and patient. The G-tube can serve to passively decompress the stomach and to provide feeding access. The G-tube includes an inflatable balloon to help secure the G-tube inside the patient. Typically G-tubes have poor functionality with respect to gastric decompression and do not allow for suctioning.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

Provided herein is a gastrostomy tube that may comprise a proximal portion and a distal portion, wherein the tube has at least one lumen that extends therethrough. The proximal portion may comprise a first port operably coupled to the proximal portion and configured to at least provide for suction of contents from a stomach of a patient. The proximal portion may also comprise a second port operably coupled to the proximal portion. A third port may be operably coupled to the proximal portion and may be configured to provide fluid to an inflatable balloon. The distal portion may comprise a plurality of first openings operably connected to the first port and configured to deliver and/or remove contents from the stomach of the patient. The distal portion may also comprise at least one second opening operably connected to the second port and configured to cooperate with the second port of the proximal portion to provide a sump-type function. The distal portion may comprise a curved portion that has a gradual bend with curvature ranging from 45°-135°. The curved portion directs a distal end of the tube radially outward and away from a longitudinal axis of the tube.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is a component diagram illustrating an example implementation of a decompressive gastrostomy tube (G-tube) of this disclosure positioned within a patient's stomach.

FIGS. 2A, 2B, 2C, and 2D are component diagrams illustrating various implementations of one or more portions of the decompressive G-tube of this disclosure.

FIGS. 3A and 3B are cross-sectional views of implementations of one or more portions of the decompressive G-tube shown in FIG. 1.

FIG. 4 is a component diagram illustrating another example implementation of a decompressive G-tube of this disclosure positioned within a patient's stomach.

FIGS. 5A and 5B are component diagrams illustrating various implementations of one or more portions of the decompressive G-tube of this disclosure.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

Existing gastrostomy tubes may have less than desired functionality with respect to gastric decompression and also present placement, leakage, and securement problems. For example, nasogastric decompressive tubes (e.g., Salem sumps) may be used to provide for gastric decompression, but they require an uncomfortable bedside procedure to place. Additionally, these nasogastric decompressive tubes often result in patient dissatisfaction. Sometimes, patients have an existing gastrostomy site that allows for access to the stomach. But, existing gastrostomy tubes do not function very well when used for gastric decompression because they do not safely allow for suctioning. Additionally, placing decompressive Salem-type tubes into the gastrostomy site can be difficult to secure to the patient and can result in leakage around the gastrostomy site.

The decompressive G-tube disclosed herein addresses these issues and provides improved performance and functionality when compared to existing G-tubes. For example, the decompressive G-tube of this disclosure has the ability to have a sump-type function. As will be explained in more detail below, the decompressive G-tube can provide for suctioning while eliminating the need for nasogastric tube placement. Moreover, the decompressive G-tube of this disclosure can reduce, and potentially eliminate, leaking of traditional Salem-type drains into the gastrostomy site as well as provide for improved securement of the G-tube within the patient. It will be appreciated that the G-tube provided by this disclosure can be safely inserted into the gastro-intestinal tract and correspondingly used.

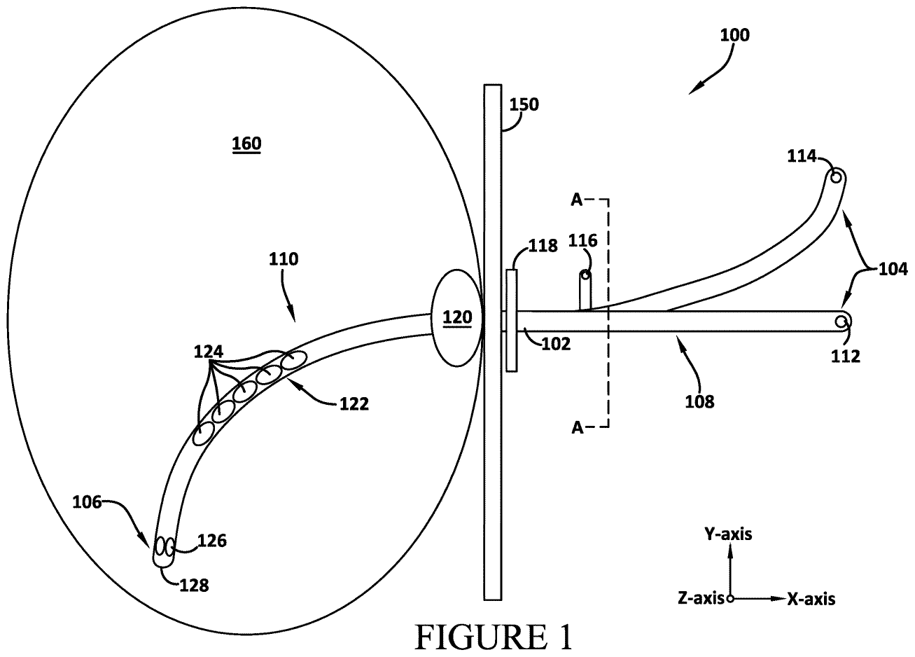

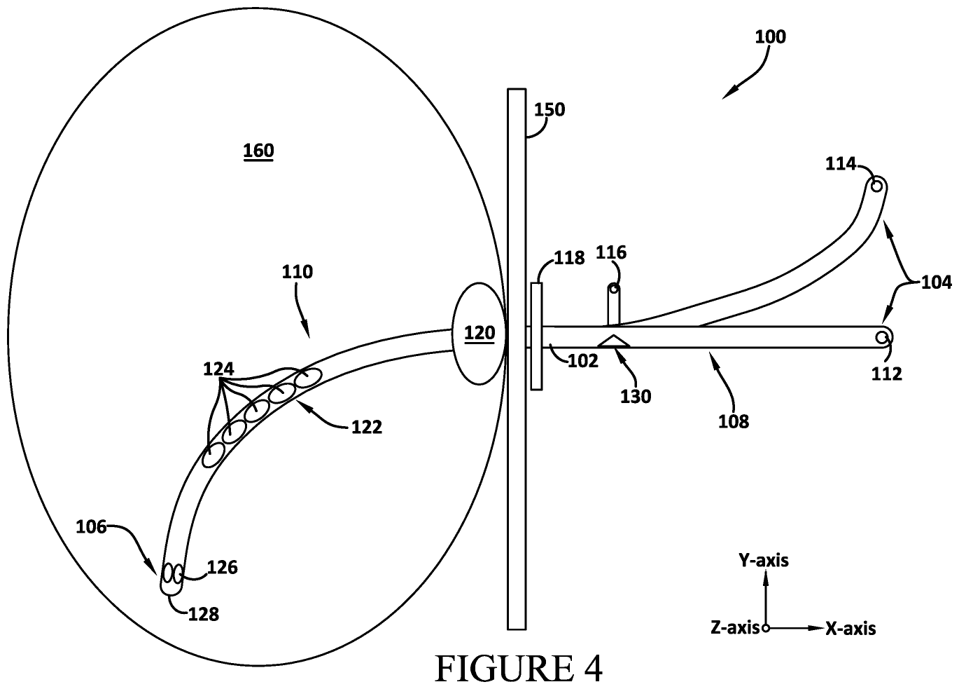

FIGS. 1 and 4 show a gastrostomy tube (G-tube) 100 for enteral nutrition and gastric decompression that may be partly disposed inside a patient's stomach 160. The G-tube 100 may comprise a tube 102 with at least one lumen formed therethrough. The tube 102 may extend from a proximal end 104 to a distal end 106 with at least one lumen extending continuously therethrough from the proximal end 104 to the distal end 106. As shown in FIG. 1, the G-tube 100 can be placed through a patient's abdominal wall 150 such that a proximal portion 108 of the G-tube 100 is disposed outside of the target patient's abdomen (i.e., externally disposed) and a distal portion 110 of the G-tube 100 is disposed inside the patient's stomach 160 (i.e., internally disposed).

In some implementations, the tube 102 can be comprised of a material that provides the tube 102 with high elastic restoring force, such as an elastic plastic material, so that the tube 102 returns to its original shape after temporary deformation. As an example, suitable materials for the tube 102 include polyurethane, silicone, and other similar materials. The tube 102 can have sufficient strength and stability to be flexible, yet not suffer kinks and closure of the lumen. At the same time, the wall thickness of the tube 102 may be as small as possible to provide a large diameter for the continuous lumen to facilitate delivery of product into the patient's stomach 160 or the removal of contents from the patient's stomach 160 using the G-tube 100. In some implementations, the tube 102 can have a soft upper layer and surface to avoid injury when placing the G-tube 100 inside a patient's stomach 160. As an example, the surface of the tube 102 can be smooth on contact with water or moisture, such as when the tube 102 is inserted into the patient's body (e.g., stomach 160).

In some implementations, the tube 102 may comprise a plurality of lumens in order to facilitate enteral nutrition with simultaneous relieving of the stomach. In some implementations, the tube 102 may comprise three lumens such as, for example, an air sump lumen, a suction lumen, and a balloon lumen. The air sump lumen can be configured to at least provide a sump-type function to the G-tube. The suction lumen can be configured to at least provide for suction of gastric juices from the stomach of a patient. The balloon lumen can be configured to provide fluid to inflate a balloon and to remove fluid to deflate the balloon. The air sump lumen, suction lumen, and balloon lumen are not limited to any particular size or shape.

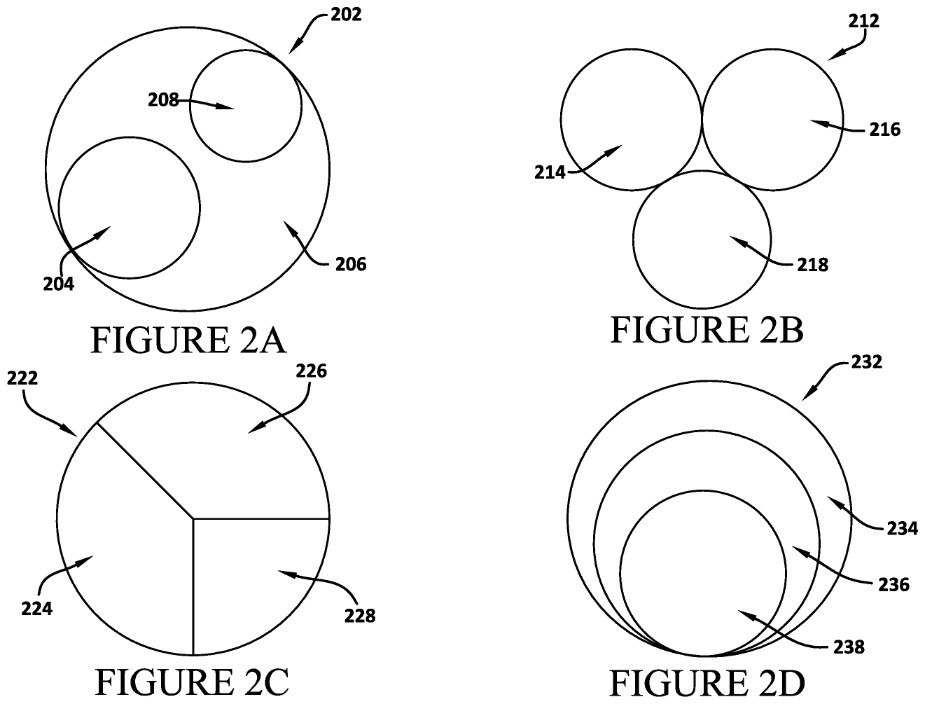

Referring now to FIGS. 2A-2D, several implementations of a tube 202, 212, 222, 232 each comprising a plurality of lumens are illustrated. In these implementations, the plurality of lumens comprise an air sump lumen, a suction lumen, and a balloon lumen. In some implementations, the tube 202 can comprise an air sump lumen 204, a suction lumen 206, and a balloon lumen 208 formed by disposing one or more separate chambers inside another tube, as shown in FIG. 2A. In other implementations, the tube 212 can comprise an air sump lumen 214, a suction lumen 216, and a balloon lumen 218 formed from multiple tubes that are joined together side-by-side, as shown in FIG. 2B. In these implementations, the multiple tubes can comprise at least a balloon tube and a gastric tube that are joined together. For example, the balloon tube can be operably coupled and in fluid communication with a balloon port and the gastric tube can be operably coupled and in fluid communication with a gastric port or suction port. In yet other implementations, the tube 222 can comprise an air sump lumen 224, a suction lumen 226, and a balloon lumen 228 formed by creating separate chambers within a larger tube, as shown in FIG. 2C. In still other implementations, the tube 232 can comprise an air sump lumen 234, a suction lumen 236, and a balloon lumen 238 formed from multiple tubes that are disposed in a nested arrangement with the tubes being non-concentric, as shown in FIG. 2D. In this particular implementation, the tubes are directly or indirectly attached to one another at a common location such that the tubes have a tangent point/region in common. In FIG. 2D the balloon lumen 238 is the smallest, innermost lumen of the tube 232. But, this disclosure is not limited to any particular configuration or arrangement of tubes and associated lumens. For example, in other implementations the balloon lumen may be the outermost lumen or the balloon lumen may be disposed between the air sump lumen and suction lumen.

The G-tube 100 provided herein is not limited to any particular size. In some implementations, the G-tube 100 can range in size from 12-24 French (12, 14, 16, 18, 20, 22, 24) to correlate with the size of the gastrostomy tract. It may be advantageous to manufacture the G-tubes 100 in commercially recognized sizes to mitigate leaking and to prevent the gastrostomy tract shrinking in size during use. In some implementations, the G-tubes 100 can be selected based on the size of the gastrostomy site. In some implementations, the length of the G-tube 100, and in particular the length of an intra-gastric portion, can be adjusted to accommodate patients of various sizes. As an example, physically smaller patients can have smaller stomachs and the size of the intra-gastric portion can be tailored to the G-tube diameter to provide adequate functionality, but not excess length for individual patients. In some of these implementations, it is conceivable that a short segment of intra-gastric portion may be needed (e.g., approximately 10 cm), which can be relatively universal among patients. It will appreciated that different lengths of intra-gastric tube may need to be considered.

In some implementations, the proximal portion 108 of the G-tube 100 can comprise one or more of a first port 112, a second port 114, a third port 116, an external retaining member 118, and a tube orientation indicia 130. The first port 112, second port 114, and third port 116 can serve different purposes. In some non-limiting examples, the first port 112 can comprise a suction port and/or a feeding port, the second port 114 can comprise an air sump port, and the third port 116 can comprise a balloon port. In these examples, the third port 116, or balloon port, and second port 114, or air sump port, each can be operably coupled to the proximal portion 108 of the tube 102 in fluid communication with the tube 102 (i.e., or other tubes disposed with the tube 102).

In some implementations, the proximal portion 108 of the G-tube 100 can comprise a first port 112 disposed at or near the proximal end 104 of the tube 102. As will be described in more detail below, the first port 112 can be connected to a plurality of first openings 124 disposed in the distal portion 110 of the tube 102 via a first lumen (e.g., 206, 216, 226, 236 of FIGS. 2A-2D) such that the first port 112 and first openings 124 are in fluid communication with each other.

In some implementations, the first port 112 can comprise a suction port. The suction port is configured to be disposed outside the patient's stomach 160 (e.g., externally disposed) when the G-tube 100 is placed inside the patient's stomach. The suction port can be operably connected to and in fluid communication with fluid openings, which may be disposed within the patient's stomach 160 (e.g., internally disposed). The suction port can be used to suck gastric juices from the stomach 160 via a suction lumen (e.g., 206, 216, 226, 236 of FIGS. 2A-2D) of the tube 102. In some implementations, the first port 112 is a suction port that can be used to remove/extract contents from the stomach 160 of the patient. In some non-limiting examples, the first port 112 is alternatively, or additionally, a feeding port that can be used to introduce contents into the patient as part of nutritional therapy and/or drug therapy.

In some implementations, the proximal portion 108 of the G-tube 100 can comprise a second port 114 disposed at or near the proximal end 104 of the tube 102. As will be described in more detail below, the second port 114 can be connected to at least one second opening 126 disposed in the distal portion 110 of the tube 102 via a second lumen (e.g., 204, 214, 224, 234 of FIGS. 2A-2D) such that the second port 114 and second opening 126 are in fluid communication with each other.

In some implementations, the second port 114 can comprise an air sump port. The air sump port is configured to be disposed outside the patient's stomach 160 (e.g., externally disposed) when the G-tube 100 is placed inside the patient's stomach. The air sump port can be operably connected to and in fluid communication with an air sump opening, which may be disposed within the patient's stomach 160 (e.g., internally disposed), to provide sump-type function. In some non-limiting examples, the second port 114 is an air sump port that can be operably connected to and in fluid communication with at least one air sump opening via an air sump lumen (e.g., 204, 214, 224, 234 of FIGS. 2A-2D) of the tube 102 to provide the sump-type function. In some implementations, the second port 114 can be covered with a removable cap (not shown) such as when the G-tube 100 is used for feeding.

In some implementations, the proximal portion 108 of the G-tube 100 can comprise a third port 116. In some implementations, the third port 116 can comprise a balloon port. The balloon port can be used to inflate a balloon 120 inside the target patient's stomach 160 to help secure (e.g., at least temporarily) the G-tube 100 at a desired position with respect to the patient. In some implementations, the balloon port is configured to be disposed outside the patient's stomach 160 (e.g., externally disposed) when the G-tube 100 is placed inside the patient's stomach. The balloon port may be operably connected to and in fluid communication with a balloon 120, which may be disposed within the patient's stomach 160 (e.g., internally disposed), to introduce fluid into the balloon 120 and/or remove fluid from the balloon 120. In some implementations, the third port 116 is a balloon port that can be used to introduce fluid, such as a gas or liquid (e.g., distilled/sterile water), into the balloon 120 disposed in the patient's stomach 160 through a balloon lumen (e.g., 208, 218, 228, 238 of FIGS. 2A-2D) to inflate the balloon 120. In some implementations, the balloon port can also be used to remove the fluid, such as a gas or liquid (e.g., distilled/sterile water), from the balloon 120 through the balloon lumen (e.g., 208, 218, 228, 238 of FIGS. 2A-2D) of the tube 102 in order to deflate the balloon 120. Deflating the balloon 120 may allow for removal of the G-tube 100 from inside the patient's stomach 160.

In some implementations, the first port 112 and second port 114 can each extend from the proximal end 104 of the tube 102 with the first and second ports 112, 114 spaced apart such that the first and second ports 112, 114 form a Y-shaped configuration.

In some implementations, the G-tube 100 can comprise an external retaining member 118. The external retaining member 118 can be used to secure the G-tube 100 to the patient through cooperation with the inflated balloon 120. As an example, the external retaining member 118 can be disposed outside the patient's stomach and in contact with the outer abdominal wall 150 while the inflated balloon 120 is disposed inside the patient's stomach and in contact with the stomach wall. In some implementations, the external retaining member 118 can be adjustable to account for differences in abdominal wall thickness between patients. As an example, the external retaining member 118 can be adjusted by repositioning the external retaining member 118 at a new position along the tube 102 to provide physical contact (i.e., surface engagement) between the external retaining member 118 and the abdominal wall 150. In this manner, the external retaining member 118 can safely and reliably hold the G-tube 100 in a stable position within the patient's body because the external retaining member 118 mitigates movement of the G-tube 100 to the inside of the patient.

In some implementations, the external retaining member 118 can be slidably engaged with the tube 102 to facilitate repositioning of the external retaining member 118 along the tube 102 to accommodate varying thicknesses of a patient's abdominal wall 150. In other implementations, the external retaining member 118 can be a removable clamp or fastening mechanism that can be detached from a first position and moved to a second position on the tube 102 where the external retaining member 118 is reattached. In one implementation, a disk or other type of fastener can be secured on the G-tube 100 against the outer abdominal wall 150 to mitigate movement of the G-tube 100 to the inside of the patient.

In some implementations, the G-tube 100 can be a MIC-KEY gastrostomy type that is sized to account for the specific thickness of the patient's abdominal wall. In these implementations, the MIC-KEY gastrostomy tubes are each sized to accommodate a specific individual patient.

In some implementations, the distal portion 110 of the G-tube 100 can comprise one or more of the balloon 120, a plurality of first openings 124 for product delivery and/or removal, a curved portion 122, at least one second opening 126, and a tip 128.

In some implementations, the distal portion 110 can comprise a balloon 120. The balloon 120 is correspondingly attached to the distal portion 110 of the G-tube 100, which is placed inside the patient. In some implementations, the balloon 120 partially wraps around the perimeter of the distal portion 110 of the tube 102 leaving at least a portion, or a part of, the tube 102 uncovered by the balloon 120 (e.g., when the balloon is at least partially inflated) and exposed. In other implementations, the balloon 120 completely wraps around the perimeter of the distal portion 110 of the tube 102 such that distal portion 110 of the tube 102 is entirely covered by the balloon 120. In some implementations, the balloon 120 can be inflated and disposed against the inside of the abdominal wall 150 to help hold the G-tube 100 in place through cooperation with the external retaining member 118 (e.g., stop) that is disposed against the outside of the abdominal wall 150. In these implementations, the inflated balloon 120 inside the patient's stomach 160 can help secure the G-tube 100 because the balloon 120 mitigates movement of the G-tube 100 to the outside of the patient.

In some implementations, the distal portion 110 of tube 102 can comprise a plurality of first openings 124. In some implementations, the first openings 124 can comprise fluid openings that may be used to deliver products (e.g., nutrition, water, medicine, etc.) into the patient's stomach 160 and/or to remove products (e.g., food, waste, etc.) from the patient's stomach 160. The first openings 124 can have a size configured to reliably facilitate ingress to the stomach 160 and egress from the stomach 160.

In some implementations, the first openings 124 are generally disposed over a portion of the tube 102 proximate to the distal end 106. In some implementations, the first openings 124 are arranged in an organized pattern. As an example, the first openings 124 can be evenly spaced apart on the distal portion 110 of the tube 102, wherein the first openings 124 are also arranged offset over the circumference. In other implementations, the first openings 124 can be arranged randomly or semi-randomly over a portion of the tube 102.

In this implementation, the distal portion 110 can comprise at least one second opening 126. In some implementations, the at least one second opening 126 comprises an air sump opening that is disposed inside the patient's stomach 160 when the G-tube 100 is placed in the patient. The second opening 126, which may be an air sump opening, cooperates with the second port 114, which may be an air sump port, disposed outside the patient's body to produce a decompressive G-tube 100 that has the ability to provide sump-type function and provides for suctioning.

In some implementations, the distal end 106 can include a soft tip 128 to prevent injury to a patient when inserting the G-tube 100 into the patient's stomach 160 and when securing the G-tube 100 in place in the patient's stomach 160. As an example, the tip 128 can be made of a soft, flexible material like plastic. In some implementations, the tip 128 can be rounded along the edges to prevent injury to the patient, such as injury to patient tissue.

In some implementations, the distal portion 110 of the tube 102 can comprise a curved portion 122. The curved portion 122 can comprise a gradual bend (e.g., gentle curve) in the tube 102 in the range of about 45°-135° and, more particularly, in the range of about 85°-95°. In some implementations, the curved portion 122 comprises a gradual bend in the tube 102 of approximately 90°. The curved portion 122 directs the distal end 106 of the tube 102 radially outward and away from a longitudinal axis of the tube 102. The curved portion 122 generally comprises a gentle curve or gradual bend in order to produce rounded edges (i.e., smooth surfaces) rather than sharp points/edges that could cause injury to patient tissue and/or to avoid any kinking or lumen closure of the tube 102 resulting from a sharp curve or steep bend.

The curved portion 122 changes the directionality of the distal end 106 of the tube 102 relative to the proximal end 104 based on the degree of curvature of the curved portion 122. Additionally, the curved portion 122 changes the orientation of the distal portion 110, in particular features on the distal portion 110, because those features are now displaced two-dimensionally (x-direction and y-directional) compared to a straight, linear G-tube.

In some implementations, the curved portion 122 may be oriented with respect to the third port 116, which may comprise a balloon port. For example, the curved portion 122 and the third port 116 can be disposed in a plane with an angular offset relative to each other as defined by an angle, α, between the curved portion 122 and the third port 116. This angle, α, defined between the curved portion 122 and the third port 116 can be between 0° and 180°.

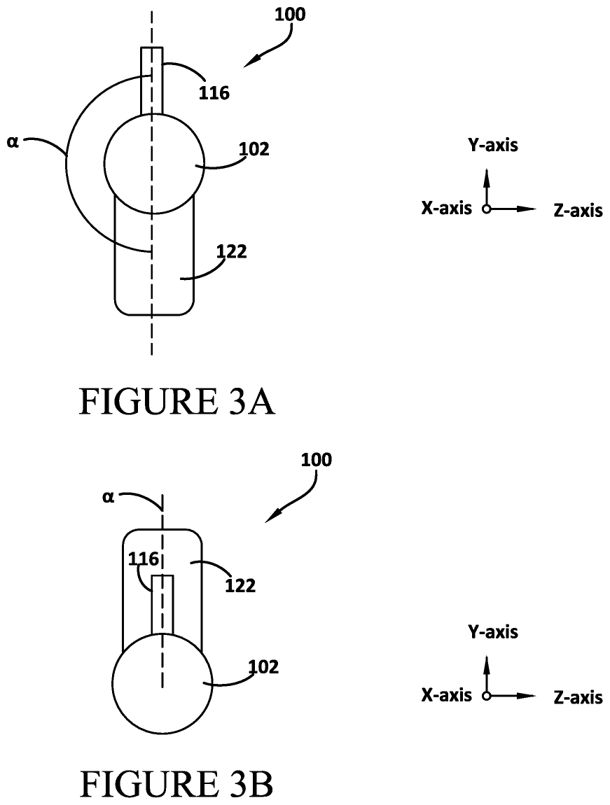

Referring now to FIGS. 3A-3B, there are cross-sectional views of the G-tube of FIG. 1 taken along line A-A that show the curved portion 122 oriented with respect to the third port 116. As an example, FIG. 3B shows an implementation in which both the curved portion 122 and the third port 116 are disposed in the yz-plane with no angular offset relative to each other such that the angle, α, defined between the curved portion 122 and the third port 116 is 0°. In instances where the angle α is 0°, the curved portion 122 and the third port 116 are said to be in phase relative to one another. As another example, FIG. 3A shows an implementation in which the curved portion 122 and the third port 116 are disposed in the yz-plane but with an angular offset relative to each other such that the angle, α, defined between the curved portion 122 and the third port 116 is 180°. In instances where the angle α is 180°, the curved portion 122 and the third port 116 are said to be out of phase relative to each other.

In some implementations, the orientation/directionality of the curved portion 122 of the tube 102 may be indicated by tube orientation indicia 130 disposed on the proximal portion 108 of the tube 102. FIG. 4 shows the curved portion 122 disposed in alignment with the tube orientation indicia 130. It will be appreciated that the tube orientation indicia 130 can be used to indicate any orientation/directionality of the curved portion 122 relative to the longitudinal axis of the tube 102 with a high level of accuracy. It will be appreciated that the tube orientation indicia 130 may be disposed on the tube 102 to indicate the orientation/directionality of the curved portion 122 even in the implementations shown in FIGS. 3A-3B, but is not required. If the tube orientation indicia 130 is included in an implementation comprising a curved portion 122 oriented with respect to the third port 116, the tube orientation indicia 130 may be used to verify the orientation/directionality of the curved portion 122. In this implementation, the curved portion 122 may direct the distal end 106 of the tube 102 to extend in the same relative radial direction (e.g., x-, y-, z-dimensions) as the tube orientation indicia 130 with respect to the longitudinal axis of the tube 102.

In some implementations, the curved portion 122 is a predefined feature of the tube 102 having a degree of curvature formed during manufacturing. The curved portion 122, and in particular the tube 102, is generally composed of a material that provides the curved portion 122 with high elastic restoring force (e.g., elastic plastic material) so that the curved portion 122 returns to its original shape after temporary deformation. As an example, the curved portion 122 can be deformed/deflected from its natural, resting position (e.g., first position) for a period of time while it temporarily assumes any one of a variety of second positions, which are different from the resting position (e.g., first position). But, due to the high elastic restoring force of the G-tube 100, the curved portion 122 eventually returns to its resting position or first position. This beneficial feature reduces kinking and tube closure, and promotes restoration of the curved portion 122 after deformation.

The curved portion 122 can be particularly advantageous with respect to placement of the G-tube 100. As an example, the curved portion 122 can ensure the G-tube 100 remains intra-gastric and does not enter into the duodenum. In some implementations, if the distance from the gastric tract to the pylorus is short, a curved portion 122 having approximately a 90° gradual bend can be disposed just beyond the balloon 120 to keep the G-tube 100 positioned intra-gastric as long as the directionality of the curved portion 122 is known during G-tube placement to position the curved portion 122 away from the pylorus of the stomach as needed.

In some implementations, the proximal portion 108 of the G-tube 100 can comprise the tube orientation indicia 130. The tube orientation indicia 130 can be included on the G-tube 100 to indicate the directionality of the curved portion 122 of the G-tube 100. In some implementations, the tube orientation indicia 130 may be aligned with the balloon port 116. In other implementations, the tube orientation indicia 130 may be aligned opposite (e.g., 180°) from the balloon port 116.

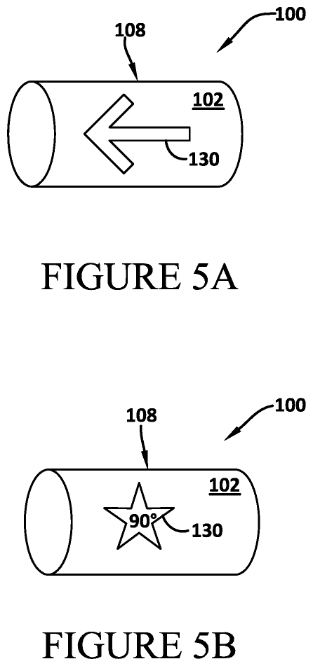

Referring now to FIGS. 5A-5B, there are illustrated several implementations of tube orientation indicia 130. The tube orientation indicia 130 can comprise printed markings included on the proximal portion 108 of the tube 102. The printed markings can be can be painted/applied to the surface of the tube 102. Alternatively, the tube orientation indicia 130 can be formed directly into the material comprising the tube 102 such as via etching. FIG. 5A shows the tube orientation indicia 130 in the form of an arrow printed on the proximal portion 108 of the tube 102. But, other types of markings can serve as the tube orientation indicia 130. As an example, FIG. 5B shows a tube orientation indicia 130 in the shape of a star including information about the degree of bend of the curved portion 122. The tube orientation indicia 130 can be color coded with certain colors corresponding to particular curvatures of the curved portion 122. During use of the G-tube 100, medical personnel can determine the orientation and directionality of the curved portion 122 (and distal portion 110) and distal end 106, while the curved portion 122 is disposed inside the patient's stomach 160, solely based on the orientation of the tube orientation indicia 130 on the G-tube 100.

A method may be devised for manufacturing and using the gastrostomy tube (G-tube) 100 described herein. In the exemplary method, the G-tube 100 comprises a tube 102 composed of a material that provides the tube 102 with high elastic restoring force, such as an elastic plastic material, so that the tube 102 returns to its original shape after temporary deformation. In this implementation, the curved portion 122 is a predefined feature of the tube 102 with a gradual bend of 45°-135° that can be formed during manufacturing. In some implementations, the curved portion 122 has a gradual bend of 85°-95° and, in one or more implementations, a gradual bend of approximately 90° that is formed during manufacturing. In yet another implementation, the G-tube 100 may be manufactured without any bend.

In this exemplary method, a user can accurately and safely direct placement of the distal portion 110 of the G-tube 100 into the patient's stomach 160 using the tube orientation indicia 130, which indicates the directionality (i.e., radial direction) of the curved portion 122. But, even with the benefit of the curved portion 122 acting in cooperation with the tube orientation indicia 130, the distal portion 110 can still be deflected by patient tissue during placement. In some implementations, deflection of the tube 102 can temporarily deform/distort the curved portion 122 causing the curved portion 122 to assume a second position that is different from its first position (i.e., manufactured position or resting position). But, due to the high elastic restoring force of the G-tube 100, the curved portion 122 eventually returns to its first position. This beneficial feature reduces kinking and tube closure, and promotes restoration of the curved portion 122 after deformation.

In this exemplary method, the G-tube 100 is secured to the patient via cooperation between the inflated balloon 120 exerting force against the inside of the abdominal wall 150 and the adjustable external retaining member 118 exerting force against the outside of the abdominal wall 150. At this point, the G-tube 100 can be virtually divided (i.e., segmented) by the patient's abdominal wall 150 into a proximal portion 108 and a distal portion 110. In this implementation, the proximal portion 108 consists of the portion of the G-tube 100 disposed outside of the patient's body (e.g., externally protruding from the abdominal wall 150) and the distal portion 110 consists of the portion of the G-tube 100 disposed inside the patient's body (e.g., internally protruding from the abdominal wall 150).

The G-tube 100 described herein offers numerous advantages over existing devices. For example, providers currently have to choose between placing a nasogastric tube in addition to placing a gastrostomy to provide gravity drainage or to place a Salem-type drain into the gastrostomy site. But, these devices provide challenges with respect to securement and drainage.

The present disclosure, on the other hand, provides a decompressive gastrostomy tube that has the ability to have a sump-type function and allows for suctioning, while eliminating the need for nasogastric tube placement. Moreover, the decompressive gastrostomy tube provided herein can mitigate leaking of traditional Salem-type drains into the gastrostomy site and eliminate associated securement issues. It is envisioned that the decompressive G-tube of this disclosure can used to care for patients with any bowel obstruction, ileus, gastroparesis, dysmotility who would benefit from gastric decompression and who already have an existing gastrostomy tube site that is well-healed. Specific examples include patients with adhesive bowel obstructions or postsurgical patients waiting for post-surgical ileus to resolve.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, At least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this disclosure. It is intended to include all such modifications and alterations in so far as they come within the scope of the appended claims or the equivalents thereof.

What is claimed is:

1. A gastrostomy tube, comprising:
a tube comprising a proximal portion and a distal portion, wherein the tube has at least one lumen that extends therethrough;
wherein the proximal portion comprises:
a first port operably coupled to the proximal portion and configured to at least provide for suction of contents from a stomach of a patient;
a second port operably coupled to the proximal portion; and
a third port operably coupled to the proximal portion and configured to provide fluid to an inflatable balloon; and
wherein the distal portion comprises:
a plurality of first openings operably connected to the first port and configured to deliver and/or remove contents from the stomach of the patient;
at least one second opening operably connected to the second port and configured to cooperate with the second port of the proximal portion to provide a sump-type function; and
a curved portion having a gradual bend with curvature ranging from 45°-135°, wherein the curved portion directs a distal end of the tube radially outward and away from a longitudinal axis of the tube.

2. The gastrostomy tube of claim 1, wherein the curved portion has the gradual bend in the curvature range of 85°-95°.

3. The gastrostomy tube of claim 1, wherein the curved portion is oriented with respect to the third port, wherein:
(i) the curved portion and the third port are disposed in a two-dimensional plane and an angle, α, defined between the curved portion and the third port is about 0° such that the curved portion and the third port have no angular offset relative to each other and are in phase; or
(ii) wherein the curved portion and the third port are disposed in the two-dimensional plane and the angle, α, defined between the curved portion and the third port is about 180° such that the curved portion and the third port have an angular offset relative to each other and are out of phase.

4. The gastrostomy tube of claim 1, wherein the proximal portion further comprises:
a tube orientation indicia disposed on the proximal portion of the tube.

5. The gastrostomy tube of claim 4, wherein the curved portion is oriented with respect to the tube orientation indicia, and wherein the tube orientation indicia is configured to indicate directionality/orientation of the curved portion.

6. The gastrostomy tube of claim 1, wherein the tube is comprised of an elastic plastic material with high elastic restoring force such that the curved portion is configured to be deflected from a first position to a second position and to return to the first position after force is no longer applied to the curved portion.

7. The gastrostomy tube of claim 1, wherein the proximal portion further comprises:
an adjustable external retaining member that is configured to be repositioned along the proximal portion of the tube.

8. The gastrostomy tube of claim 7, wherein the distal portion further comprises:
a balloon attached to the distal portion, wherein the balloon is configured to cooperate with the external retaining member to secure the gastrostomy tube to the patient.

9. The gastrostomy tube of claim 1, wherein the first port comprises a suction port and/or a feeding port, wherein the second port comprises an air sump port, and wherein the third port comprises a balloon port.

10. A gastrostomy tube, comprising:
a tube having a proximal portion and a distal portion, wherein the tube includes a plurality of lumens extending therethrough from the proximal portion to the distal portion;
wherein the proximal portion comprises:
a first port operably coupled to the proximal portion and configured to at least provide for suction of contents from a stomach of a patient;
a second port operably coupled to the proximal portion;
an adjustable external retaining member that is configured to be repositioned along the proximal portion of the tube; and
a third port operably coupled to the proximal portion and configured to provide fluid to an inflatable balloon; and
wherein the distal portion comprises:
a plurality of first openings operably connected to the first port via a first lumen and configured to deliver and/or remove contents from the stomach of the patient;
at least one second opening operably connected to the second port via a second lumen and configured to cooperate with the second port of the proximal portion to provide a sump-type function; and
a curved portion having a gradual bend with curvature ranging from 45°-135°, wherein the curved portion directs a distal end of the tube radially outward and away from a longitudinal axis of the tube.

11. The gastrostomy tube of claim 10, wherein the curved portion has the gradual bend in the curvature range of 85°-95°.

12. The gastrostomy tube of claim 10, wherein the curved portion is oriented with respect to the third port, wherein:
(i) the curved portion and the third port are disposed in a two-dimensional plane and an angle, α, defined between the curved portion and the third port is about 0° such that the curved portion and the third port have no angular offset relative to each other and are in phase; or
(ii) wherein the curved portion and the third port are disposed in the two-dimensional plane and the angle, α, defined between the curved portion and the third port is about 180° such that the curved portion and the third port have an angular offset relative to each other and are out of phase.

13. The gastrostomy tube of claim 10, wherein the proximal portion further comprises:
a tube orientation indicia disposed on the proximal portion of the tube, wherein the curved portion is oriented with respect to the tube orientation indicia, and wherein the tube orientation indicia is configured to indicate directionality/orientation of the curved portion.

14. The gastrostomy tube of claim 10, wherein the first port comprises a suction port and/or a feeding port, wherein the second port comprises an air sump port, and wherein the third port comprises a balloon port.

15. The gastrostomy tube of claim 10, wherein the tube is comprised of an elastic plastic material with high elastic restoring force such that the curved portion is configured to be deflected from a first position to a second position and to return to the first position after force is no longer applied to the curved portion.

16. The gastrostomy tube of claim 10, wherein the distal portion further comprises:
a balloon that is attached to the distal portion, wherein the balloon is configured to cooperate with the external retaining member to secure the gastrostomy tube to the patient.

17. A gastrostomy tube, comprising:
a tube having a proximal portion and a distal portion, wherein the tube includes a plurality of lumens extending therethrough from the proximal portion to the distal portion;
wherein the proximal portion comprises:
a first port operably coupled to the proximal portion and configured to at least provide for suction of contents from a stomach of a patient;
a second port operably coupled to the proximal portion;
an adjustable external retaining member that is configured to be repositioned along the proximal portion of the tube;
a tube orientation indicia disposed on the proximal portion of the tube; and
a third port operably coupled to the proximal portion and configured to provide fluid to an inflatable balloon; and
wherein the distal portion comprises:
a plurality of first openings operably connected to the first port and configured to deliver and/or remove contents from the stomach of the patient;
at least one second opening operably connected to the second port and configured to cooperate with the second port of the proximal portion to provide a sump-type function; and
a curved portion having a gradual bend with curvature ranging from 45°-135°, wherein the curved portion directs a distal end of the tube radially outward and away from a longitudinal axis of the tube, wherein the curved portion is oriented with respect to the tube orientation indicia which indicates directionality/orientation of the curved portion,
wherein the tube is comprised of an elastic plastic material with high elastic restoring force such that the curved portion is configured to be deflected from a first position to a second position and to return to the first position after force is no longer applied to the curved portion.

18. The gastrostomy tube of claim 17, wherein the curved portion has the gradual bend in the curvature range of 85°-95°.

19. The gastrostomy tube of claim 17, wherein the curved portion is oriented with respect to the third port, wherein:
(i) the curved portion and the third port are disposed in a two-dimensional plane and an angle, α, defined between the curved portion and the third port is about 0° such that the curved portion and the third port have no angular offset relative to each other and are in phase; or
(ii) wherein the curved portion and the third port are disposed in the two-dimensional plane and the angle, α, defined between the curved portion and the third port is about 180° such that the curved portion and the third port have an angular offset relative to each other and are out of phase.

20. The gastrostomy tube of claim 17, wherein the first port comprises a suction port and/or a feeding port, wherein the second port comprises an air sump port, and wherein the third port comprises a balloon port.
